(19) United States
(12) Patent Application Publication
(10) Pub. No.: US 2009/0233895 A1
(43) Pub. Date: Sep. 17, 2009

(54) METHOD OF TREATMENT OF BACTERIAL INFECTIONS

(75) Inventors: Olivier Petitjean, Senlis (FR); Patrick Nicolas, Paris (FR); Mohamed Kamel Louchahi, Caen (FR)

Correspondence Address: BUCHANAN, INGERSOLL & ROONEY PC, POST OFFICE BOX 1404, ALEXANDRIA, VA, 22313-1404, US

(73) Assignee: Ethypharm, St Cloud Cedex (FR)

(21) Appl. No.: 12/303,926

(22) PCT Filed: Jun. 7, 2007
(86) PCT No.: PCT/EP2007/055622

§ 371 Date: Mar. 31, 2009

Related U.S. Application Data
(60) Provisional application No. 60/804,226, filed on Jun. 8, 2006.

(51) Int. Cl. A61K 31/545; A61K 31/4025; A61P 31/04

(52) U.S. Cl. 514/200; 514/422

(57) ABSTRACT

The present invention relates to a method of treatment of bacterial infections including administering an effective amount of an oral time-dependent antibiotic to a human or warm blooded animal.

The present invention relates to a method of treatment of bacterial infections comprising administering an effective amount of an oral time-dependent antibiotic to a human or warm blood animal.

In case of a time-dependent anti-bacterial treatment the main requirement is to maintain the blood concentration of the anti-bacterial agent at a high level throughout the entire duration of the treatment, preferably above the minimum inhibitory concentration (MIC). This does not only maximize the success of the anti-bacterial treatment, but also minimizes the risk of promoting bacterial resistance. An example of an oral time-dependent (also known as concentration-independent) antibiotic are beta-lactamines, when used against gram negative Bacteria (GNB).

Beta-lactamines comprise the group of penicillins or Penames, including the homologues of Penicillin G, Penicillin M, Penicillin A, as well as the 6 alpha-penicillins, alpha-carboxy- and alpha-sulfopenicillins, amino-penicillins and oxy-iminopenicillins, the group of cephemes comprising the cephalosporines, oxacephemes, carbacephemes, isocephemes, azacephemes and phosphocephemes, the group of Penemes comprising the carbapenemes and the oxapenemes, the group of monocyclic monobactames and the group of the beta-lactamase inhibitors.

Beta-lactamines are antibiotics that are widely used, especially in the treatment of otorhinolaryngologic, broncho-pulmonary and urinary infections. Beta-lactamines are particularly widely used against both gram positive (GPB) and gram negative bacteria (GNB).

It is known that in order to approach a maximum antibacterial effect, the plasma concentrations of time-dependent antibiotics should be maintained above the MIC for 60%-70% of the dosing interval.

Conventionally, strains are classified in three categories, each depending on the susceptibility of the strains versus the antibiotic. The antimicrobial susceptibility testing is based on in vitro standardized techniques that give two pharmacological breakpoints.

Breakpoints are discriminatory antimicrobial concentrations integrating the drug potency against potential pathogens with the pharmacokinetics of the antimicrobial. Breakpoints are used in the interpretation of results of susceptibility testing to define the three following bacterial categories:
susceptible: strains with MIC below the lower breakpoint. With a susceptible micro-organism, the antimicrobial treatment leads to a high likelihood of therapeutic success.
intermediate: strains with MIC between the lower and the upper breakpoints. With an intermediate micro-organism, the antimicrobial treatment leads to indeterminate therapeutic outcome.
resistant: strains with MIC above the upper breakpoint. With a resistant micro-organism, the antimicrobial treatment leads to a high likelihood of therapeutic failure.

This classification is presented in the following figure:

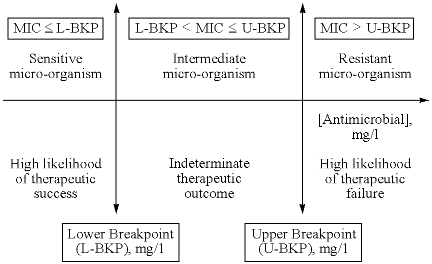

Time-dependent antibiotics are known to often have relatively short biological half-lives, e.g. 30 to 60 minutes. That is why immediate release pharmaceutical compositions of oral time-dependent antibiotics have to be administered in timely reduced intervals, e.g. every eight hours, in order to guarantee an efficient treatment. But still, the plasma concentrations of time-dependent antibiotics decrease rapidly below the MIC so that it is not possible to maintain the plasma concentrations of time-dependent antibiotics above the MIC for 60%-70% of the dosing interval, whenever MIC are close to the breakpoint values.

In the case of beta-lactamines, to the knowledge of the inventors, there is only one modified release pharmaceutical composition, that of cefaclor. Such a modified release composition is intended for decreasing the administration frequency and to obtain thereby a better patient compliance with the prescribed regimen, as resulted from standard bioequivalence studies.

As mentioned in its SPC (summary of product characteristics), modified release cefaclor is usually administered in intervals of about 12 hours, i.e. twice daily, while 8-hour intervals are required with immediate release cefaclor. However, even though the patient compliance is usually increased, the reduction of the administration frequency has a detrimental effect on the maintenance of cefaclor plasma concentrations above the MIC because of evident insufficiency of the sustained effect whenever MIC are close to the breakpoints values.

The present inventors have found that the importance of maintaining the time-dependent antibiotic plasma concentrations above the MIC becomes particularly apparent when it comes to the treatment of strains which are qualified as intermediate strains according to the antimicrobial susceptibility test. Thereafter, strains which are qualified as intermediate in the antimicrobial susceptibility test are called intermediate strains.

Against intermediate strains, the anti-bacterial treatment with conventional immediate or modified release oral time-dependent antibiotic often does not allow maintaining the plasma concentrations above the MIC for a sufficient period of time, i.e. 60%-70% of the dosing interval, thus leading to potential therapeutic failure.

The present invention therefore aims at reducing the risk of therapeutic failure in the antibacterial treatment, especially with respect to intermediate bacteria strains.

The present invention provides for a method of treatment of bacterial infections comprising the administration to a human being or a warm blood animal of an effective amount of an oral time-dependent antibiotic, wherein said time-dependent antibiotic has an apparent elimination half-life of at least 90 minutes and the dosing interval is between 6 and 12 hours, preferably between 8 and 12 hours for optimal therapeutic compliance.

The elimination half-life of a drug refers to the time required for the concentration of drug in plasma to decrease by half. When determined experimentally by measuring drug concentration in plasma samples drawn at various and successive times after drug intake, this parameter is named apparent elimination half-life.

In a preferred embodiment the apparent elimination half-life is at least 100 minutes.

The amount of time-dependent antibiotic is adjusted so as to maintain the plasma concentrations of time-dependent antibiotic above the MIC of the strain that is responsible for the infection for at least 60% of the dosing interval. Preferably, the plasma concentrations of time-dependent antibiotic are above the MIC for at least 80% of the dosing interval.

In one embodiment the plasma concentrations of time-dependent antibiotic are at least twice the MIC of the strain that is responsible for the infection for at least 60% of the dosing interval.

In a preferred embodiment the plasma concentrations of time-dependent antibiotic are at least twice the MIC of the strain that is responsible for the infection for at least 80% of the dosing interval.

The dosing interval between two doses is between 6 and 12 hours, preferably between 8 and 12 hours.

The present method of treatment can be carried out with any time-dependent antibiotic that is suitable for oral use in humans or warm-blood animals, particularly those selected from the group comprising, tetracyclines, oxazolidinones, group A and group B streptogramins, macrolides, lincosamines beta-lactamines and mixtures thereof

Examples of suitable tetracylines include chlortetracycline, oxytetracycline, tetracycline, demeclocycline, doxycycline and minocycline.

Examples of suitable streptogramins include pristinamycins, virginiamycins, mykamycins, and oestreogrycins and synergistins.

An example of a suitable oxazolidinone is linezolid.

Examples of suitable macrolides include erythromycin, flurithromycin, roxithromycin, dirithromycin (precursor of the active compound erythromycylamine), clarithromycin (or 6-methoxy-erythromycin), azithromycin, josamycin, spiramycin, carbomycin, miocamycin.

An example of a suitable lincosamine is clindamycin, or lincomycin.

For the purpose of the present invention, the reference to any suitable time-dependent antibiotic is to be understood as to include its base form, its pharmaceutically acceptable salts and esters, any polymorphic form thereof, as well as racemic or enantiomeric forms thereof.

The use of the above cited classes of time-dependent antibiotics in the method of the invention is particularly advantageous since none of them has a major post antibiotic effect, i.e. the antibacterial effect of the antibacterial agent (antibiotic) does not persist long after the end of the treatment. Unexpectedly and surprisingly the method of the invention allows the treatment of bacterial infections with the above cited classes of time-dependent antibiotics, despite the absence of a major post antibiotic effect.

In one embodiment the time-dependent antibiotic is selected from the group comprising oxazolidinones, lincomycin, clindamycin, macrolides, and fluoroquinolones.

In a preferred embodiment the time-dependent antibiotic is selected from beta-lactamines. They comprise the group of penicillins or Penames, including the homologues of Penicillin G, Penicillin M, Penicillin A, as well as the 6 alpha-penicillins, alpha-carboxy- and alpha-sulfopenicillins, amino-penicillins and oxy-iminopenicillins, the group of cephemes comprising the cephalosporins, oxacephemes, carbacephemes, isocephemes, azacephemes and phosphocephemes, the group of Penemes comprising the carbapenemes and the oxapenemes, the group of monocyclic monobactames and the group of the beta-lactamase inhibitors.

This family particularly comprises the following compounds, including their pharmaceutically acceptable salts, and esters, amoxicillin, ampicillin, apalcillin, bacampicillin, cefacetril, cefaclor, cefadroxil, cefalexin, cefamandole, cefapirin, cefatrizin, cefonicid, cefotiam, cefradin, ceftizoxim, cefuroxime, clavulanic acid, clemizol penicillin, clometocillin, cloxacillin, dicloxacillin, epicillin, flucloxacillin, hetacillin, loracarbef, metampicillin, oxacillin, penbenicillin, penethacillin, iodhydratepenimepicyclin, penimocyclin, pheneticillin, phenoxymethylpenicillin, pivampicillin, propicillin, tazobactam.

In a preferred embodiment, the oral beta-lactamine belongs to the group of cephalosporins including their pharmaceutically acceptable salts and esters.

Among cephalosporins usable according to the present invention, are cephalosporins of the first generation like cefaclor, cephadroxil, cephalexin, and cephradin. The preferred cephalosporin compound is cefaclor.

Cephalosporins of the second generation are also suitable for the present invention, in particular cefprozil.

Cephalosporins of the third generation can also be used in the present invention. Such compounds belong to the group of cefpodoxime, cefdinir, cefditoren, cefixime, ceftibuten, cefuroxime.

In one other embodiment of the present invention the time-dependent antibiotic is selected from the group of macrolides presenting a short half-life and their pharmaceutically acceptable salts and esters. These macrolides are for example erythromycin, josamycin, rovamycin, clarithromycin and telithromycin. Among these substances, preferred compounds are erythromycin, josamycin, rovamycin, and clarithromycin. Most preferred compound is clarithromycin.

In one embodiment the time-dependent antibiotic is selected from the fluoroquinolones, which are most often time-dependent antibiotics against Gram-positive bacteria.

This family particularly comprises the following compounds, including their pharmaceutically acceptable salts and esters: ciprofloxacin, ofloxacin, and levofloxacin. The preferred compounds among this class are levofloxacin and ofloxacin.

The present method is particularly useful in the treatment of infections caused by sensitive, moderately sensitive and/or intermediate strains, i.e. by a majority of Gram negative bacteria.

The method of the invention is particularly useful for treating infections caused by strains whose MIC are close to the lower critical value since it offers a greater likelihood of therapeutic success. With regards to intermediate strains, one can speculate a greater bacterial cure by administering higher doses of a beta-lactamine showing the pharmacokinetic profile proposed by the inventors.

Additionally, since the plasma concentrations of time-dependent antibiotic are maintained above the MIC for at least 60% of the dosing interval, the method of the invention leads to an increase of efficiency of the antibacterial treatment and therefore to shorter durations of treatment. Consequently, the method of the invention implicitly leads to better patient compliance.

The method of the invention is also very useful in the antibacterial treatment of patients with random reduced systemic absorption of time-dependent antibiotics. In fact, thanks to the administration at least 3 times a day (every 8 hours) of time-dependent antibiotics with an apparent elimination half-life of at least 90 minutes, it is possible to maintain the plasma concentrations to a level that has never been maintained with time-dependent antibiotics presenting shorter apparent elimination half-life.

In order to more fully illustrate the nature of the invention and the manner of practising the same, the following non-limiting examples are presented.

### EXAMPLES

In the following examples, the inventors compare the efficiency of conventional methods for treating bacterial infections with oral beta-lactamines to the method of the invention.

In all the examples, the oral beta-lactamine is cefaclor monohydrate supplied by Laboratoires Ethypharm (lot No PC9504260).

The bacteria stem from one strain of Escherichia coli, species that is considered to be non-constantly sensitive. The strain was obtained from M.-H. Nicolas (Hôpital Ambroise Paré, Paris, France) under the code E. coli GR2 with MIC of 1 mg/ml. The usual breakpoints with cefaclor for E. coli are ≦2 mg/l for sensitive strains and >8 mg/l for resistant strains, according to CA-SFM (Comité de l'Antibiogramme de la Société Française de Microbiologie).

The culture media is a Müller-Hinton Broth (MHB) obtained from Pasteur diagnostic (reference 69444). The starting inoculum of E. coli in the culture media is 10⁷±5% CFU/ml. The E. coli concentration is measured by nephelometry (Densimat, ref. 99535 ver. A, Biomérieux, France) and standard dilution method.

The counting of E. coli present in the culture medium was measured in vitro in a model simulating the antibacterial treatment with oral beta-lactamine in humans over 24 hours.

The model simulating the antibacterial treatment used in the examples is a modified “Hollow T-Tube”-model, originally described by Cappellety et al. (Pharmacodynamics of ceftazidime administered as continuous infusion or intermittent bolus alone or in combination with single daily-dose amikacine against Pseudomonas aeruginosa in an in vitro infection model, A.A.C., 1995, 33: 1797-1801) and modified by the inventors (Louchahi et al. A procedure to mimic human impaired kinetic profiles of antibiotics with the hollow glass T-tube in vitro pharmacodynamic model, Abstract no 2116, p. 234, 39^{th} ICAAC, Sep. 26-29, 1999, San Francisco).

### Example 1

Three different elimination half-lives were simulated:
1. t_{1/2}=45-50 min, which corresponds to a conventional immediate release form of cefaclor,
2. t_{1/2}=55-60 min, which corresponds to a conventional sustained release form of cefaclor,
3. t_{1/2}=90-100 min, which corresponds to an extended release form of cefaclor according to the present invention.

The first experiment E1.1 was carried out with t_{1/2}=45-50 min, 500 mg of cefaclor per dosage and a dosage interval of 8 hours.

The second experiment E1.2 was carried out with t_{1/2}=55-60 min, 750 mg of cefaclor per dosage and a dosage interval of 12 hours.

The third, fourth and fifth experiments E1.3, E1.4 and E1.5 respectively were carried out with t_{1/2}=90-100 min. In E1.3 500 mg of cefaclor per dosage and a dosage interval of 8 hours was used. In E1.4 and E1.5 750 mg of cefaclor were used and the dosage interval was 12 hours and 8 hours respectively.

In all experiments the period during which, after the addition of cefaclor, the concentration of cefaclor was above the MIC was measured (Δt>MIC). The result is expressed as percentage of the dosage interval.

The results of experiments E1.1-E1.5 are summarized in table 1.

FIGS. 1 and 2 illustrate the results of experiments E1.1 and E1.2 respectively.

FIG. 3 illustrates the results of experiments E1.4 and E1.5.

TABLE 1

| | E1.1 | E1.2 | E1.3 | E1.4 | E1.5 |
|---|---|---|---|---|---|
| Expected t_{1/2}, min | 45-50 | 55-60 | 90-100 | 90-100 | 90-100 |
| Observed t_{1/2}, min | 46 | 58 | 100 | 101 | 92 |
| cefaclor, mg | 500 | 750 | 500 | 750 | 750 |
| Dosage interval, h | 8 | 12 | 8 | 12 | 8 |
| Expected cₘₐₓ, | 13-15 | 8-10 | 9 | 9.5 | 11 |
| mg/l | | | | | |
| Observed cₘₐₓ, | 11.8 | 9.5 | 7.1 | 9.5 | 11 |
| mg/l | | | | | |
| Observed Δt > | 50 | 30-35 | 75 | 58 | 87 |
| MIC, % | | | | | |
| Δ UFC/ml by 24 h, | −1.5 | −1.5 | −4 | −1.5 | >−5 |
| log | | | | | |

### Example 2

Three different patients were simulated:
1. patient who is a poor absorber, i.e. with a Cmax of 4.9 mg/l,
2. patient who is a standard absorber, i.e. with a Cmax of 7.1 mg/l,
3. patient who is a good absorber, i.e. with a Cmax of 9 mg/l.

The three experiments E2.1, E2.2 and E2.3 were carried out with t_{1/2}=90-100 min, 500 mg of cefaclor per dosage and a dosage interval of 8 hours.

In all experiments the period during which, after the addition of cefaclor, the concentration of cefaclor was above the MIC was measured (Δt>MIC). The result is expressed as percentage of the dosage interval.

The variation of the bacterial count was regularly registered during 24 hours. The result is expressed as ΔUFC/ml. For every experiment, the negative sign is highlighting the decreasing count with time since it corresponds to the difference between the initial inoculum at T0 and final bacterial count at T 24 h.

The results of experiments E2.1-E2.3 are summarized in table 2.

FIG. 4 illustrates the results of experiments E2.1 to E2.3.

TABLE 2

| | E2.1 | E2.2 | E2.3 |
|---|---|---|---|
| Expected t_{1/2}, min | 90-100 | 90-100 | 90-100 |
| Observed t_{1/2}, min | 96 | 100 | 92 |
| Cefaclor, mg | 500 | 500 | 500 |
| Dosage interval, h | 8 | 8 | 8 |
| cₘₐₓ, mg/l | 4.9 | 7.1 | 9.0 |
| Δt > MIC, % | 50 | 75 | 87 |
| Δ UFC/ml by 24 h, log | −3 | −4 | −5 |

1. Method of treatment of bacterial infections comprising the administration to a human being or a warm blood animal of an effective amount of an oral time-dependent antibiotic, wherein said time-dependent antibiotic has an apparent elimination half-life of at least 90 minutes and the dosing interval is between 6 and 12 hours.

2. Method according to claim 1, wherein the dosing interval is between 8 and 12 hours.

3. Method according to claim 1, wherein the dosing interval is 8 hours.

4. Method according to claim 1, wherein the apparent elimination half-life is of at least 100 minutes.

5. Method according to claim 1, wherein the amount of time-dependent antibiotic is adjusted so as to maintain the plasma concentration of time-dependent antibiotic above the MIC of the strain that is responsible for the infection for at least 60% of the dosing interval.

6. Method according to claim 1, wherein the amount of time-dependent antibiotic is adjusted so as to maintain the plasma concentration of time-dependent antibiotic above the MIC of the strain that is responsible for the infection for at least 80% of the dosing interval.

7. Method according to claim 1, wherein the time-dependent antibiotic is selected from the group comprising, tetracyclines, streptogramines, lincosamides, oxazolidinones, macrolides, beta-lactamines, fluoroquinolones, cephalosporines and mixtures thereof.

8. Method according to claim 1, wherein the time-dependent antibiotic is selected from beta-lactamines and their pharmaceutically acceptable salts and esters.

9. Method according to claim 1, wherein the beta-lactamine is cefaclor or a pharmaceutically acceptable salt and ester thereof.

10. Method according to claim 1, wherein the time-dependent antibiotic is clindamycine or a pharmaceutically acceptable salt and ester thereof.

11. Method according to claim 1, wherein the time-dependent antibiotic is selected from oxazolidinones and their pharmaceutically acceptable salts and esters.

12. Method according to claim 1, wherein the time-dependent antibiotic is selected from macrolides and their pharmaceutically acceptable salts and esters.

13. Method according to claim 1, wherein the time-dependent antibiotic is selected from fluoroquinolones and their pharmaceutically acceptable salts and esters.

14. Method according to claim 1, wherein the time-dependent antibiotic is selected from cephalosporines and their pharmaceutically acceptable salts and esters.
